(19) United States
(12) Patent Application Publication
(10) Pub. No.: US 2016/0345831 A1
(43) Pub. Date: Dec. 1, 2016

(54) PHYSIOLOGICAL MONITORING SYSTEM

(71) Applicant: Augustine Yen LIEN, TAIPEI (TW)

(72) Inventor: Augustine Yen LIEN, TAIPEI (TW)

(21) Appl. No.: 14/725,165

(22) Filed: May 29, 2015

(51) Int. Cl. A61B 5/00; A61B 7/00; A61B 5/11; A61B 5/01

(52) U.S. Cl. CPC A61B 5/0013; A61B 5/0077; A61B 5/0022; A61B 5/7445; A61B 5/01; A61B 7/00; A61B 5/11; A61B 5/746; A61B 2562/0257; A61B 2560/0242

(57) ABSTRACT

A physiological monitoring system for monitoring a user's physiological status and/or first image includes a near-end portable monitoring module, an image sensing module and a near-end information display module. The near-end portable monitoring module senses the user's physiological status by a sensing unit to generate first sensed data, and the first communication unit transmits the first sensed data. The image sensing module receives the first sensed data through the second communication unit, and the image capturing unit selectively captures the user's first image. The near-end information display module receives the sensed data by the third communication unit, and the first display unit displays a frame corresponsive to the first sensed data. The physiological monitoring system may be applied in the fields of security surveillance, pet monitoring, home care or medical institutes.

### FIELD OF THE INVENTION

The present invention relates to the field of physiological monitoring technology, in particular to a physiological monitoring system for monitoring the physiological status and/or image of a user wearing a portable monitoring module.

### BACKGROUND OF THE INVENTION

In general, a conventional medical healthcare system allows patients to call a doctor or medical professional by an active method such as ringing an alarm bell. However, such arrangement is very inconvenient to the patients. In some special situations, the patients may be unable to call actively. The same situation may occur in a homecare environment. Although a sensing device may be installed to obtain the condition and situation of a care receiver, yet only data are transmitted, and there are still a lot of inconveniences.

In view of the aforementioned problems, the present invention provides a physiological monitoring system to improve the conventional image capturing device.

### SUMMARY OF THE INVENTION

It is a first objective of the present invention to provide a monitoring system comprising a near-end portable monitoring module and an image sensing module, so that after a user wears the near-end portable monitoring module, the image sensing module and/or the near-end information display module may receive the sensed data transmitted from the near-end portable monitoring module to monitor the user's physiological status and/or image.

A second objective of the present invention is to monitor another user's physiological status and/or image by another far-end portable monitoring module based on the aforementioned monitoring system, in addition to the near-end portable monitoring module, so as to achieve the effect of monitoring the physiological status and/or image of one or more users.

A third objective of the present invention is to provide a monitoring system comprising a near-end information display module and/or a far-end information display module, for displaying the user's physiological status and/or image, so that the user or any non-user (such as a therapist, a nurse, a caregiver) can monitor the user by the information display modules.

A fourth objective of the present invention is to provide the aforementioned monitoring system further comprising an alarm unit, such that when there is an abnormal image and/or physiological status, the alarm unit warns the user or other non-users.

A fifth objective of the present invention is to provide the aforementioned monitoring system, wherein the near-end portable monitoring module, the image sensing module and the near-end information display module may have unidirectional or bidirectional data transmission with one another through communication units in compliance with a cable communication or wireless communication specification, and stable and convenient installations may be conducted in different environments.

To achieve the aforementioned and other objectives, the present invention provides a monitoring system for monitoring at least one of a user's physiological status and first image, comprising: a near-end portable monitoring module, an image sensing module and a near-end information display module. The near-end portable monitoring module comprises a first communication unit and a first sensing unit. The near-end portable monitoring module is provided for the user to wear. The first sensing unit senses the user's physiological status to generate first sensed data. The first communication unit transmits the sensed data. The image sensing module comprises a second communication unit and an image capturing unit. The second communication unit receives the first sensed data, and the image capturing unit selectively captures a first image of the user. Wherein, the image capturing unit tracks the user by the near-end portable monitoring module to obtain the first image. The near-end information display module comprises a third communication unit and a first display unit. The third communication unit receives the sensed data and displays a frame corresponsive to the first sensed data through the first display unit.

Compared with the prior art, the present invention provides a monitoring system for monitoring one or more users who wear a portable monitoring module, and the image sensing module is provided for receiving the aforementioned user's physiological status and selectively capturing the user's image, and the display module is provided for displaying the related physiological status and image, so that the present invention can be applied extensively in different fields such as the fields of security surveillance, pet monitoring, home care, and medical institutes.

### BRIEF DESCRIPTION OF THE DRAWINGS

FIG. 1 is a schematic block diagram of a physiological monitoring system in accordance with a first preferred embodiment of the present invention;

FIG. 2 is a schematic block diagram of a physiological monitoring system in accordance with a second preferred embodiment of the present invention;

FIG. 3 is a schematic block diagram of a physiological monitoring system in accordance with a third preferred embodiment of the present invention;

FIG. 4 is a schematic block diagram of a physiological monitoring system in accordance with a fourth preferred embodiment of the present invention; and

FIG. 5 is a schematic block diagram of a physiological monitoring system in accordance with a fifth preferred embodiment of the present invention.

### DESCRIPTION OF THE PREFERRED EMBODIMENTS

The aforementioned and other objects, characteristics and advantages of the present invention will become apparent with the detailed description of the preferred embodiments and the illustration of related drawings as follows.

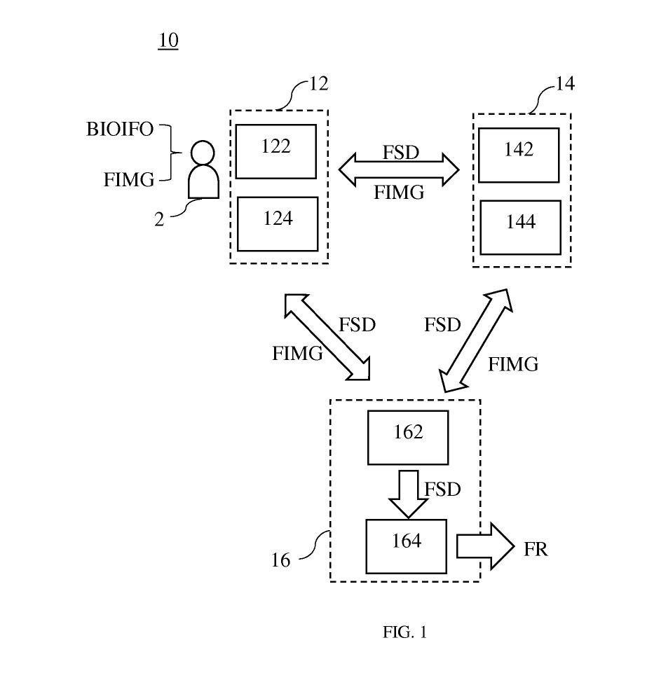

With reference to FIG. 1 for a schematic block diagram of a physiological monitoring system in accordance with the first preferred embodiment of the present invention, the physiological monitoring system 10 comprises a near-end portable monitoring module 12, an image sensing module 14 and a near-end information display module 16, and the physiological monitoring system 10 is provided for monitoring a physiological status BIOIFO and/or a first image FIMG of a user 2.

The near-end portable monitoring module 12 further comprises a first communication unit 122 and a first sensing unit 124. The near-end portable monitoring module 12 is provided for the user 2 to wear and for sensing the physiological status BIOIFO of the user 2 to generate first sensed data FSD. For example, the first sensing unit 122 is provided for sensing temperature, audio frequency, sound, humidity, brightness, movement, vital signs, physiological signal and distance. The first communication unit 122 is provided for transmitting the first sensed data FSD. For example, the near-end portable monitoring module 12 may be in form of a watch or a piece of jewelry. Wherein, the first communication unit 122 complies with a wireless communication specification and/or a cable communication specification. For the first communication unit 122 complying with the wireless communication specification, the wireless communication specification includes a short-range communication protocol and a long-range communication protocol. The short-range communication protocol may be a near field communication protocol, a Bluetooth communication protocol, a ZigBee communication protocol, a digital enhanced cordless telecommunications protocol or a wireless universal serial bus communication protocol; and the long-range communication protocol may be a wireless fidelity (Wi-Fi) communication protocol, a municipal wireless communication protocol, a general packet radio service communication protocol, a wireless broadband communication protocol, a worldwide interoperability for microwave access communication protocol, a time division duplexing communication protocol, a high speed packet access communication protocol, a high speed downlink packet access communication protocol, or a long term evolution communication protocol.

The image sensing module 14 comprises a second communication unit 142 and an image capturing unit 144. The second communication unit 142 receives the first sensed data FSD, wherein the second communication unit 142 is the same as the first communication unit 122 as described above. The image capturing unit 144 selectively captures a first image FIMG of the user 2. For example, the image capturing unit 144 is a charge coupled device (CCD) or a complimentary metal oxide semiconductor (CMOS) component, and the resolution is a standard definition (SD) resolution or a high density (HD) resolution. Wherein, the image capturing unit 144 tracks the user 2 by the near-end portable monitoring module 12 to obtain the first image FIMG.

The near-end information display module 16 comprises a third communication unit 162 and a first display unit 164. The third communication unit 162 receives the first sensed data FSD. Wherein, the third communication unit 162 is the same as the first communication unit 122 as described above. In addition, the first display unit 164 displays a frame FR corresponsive to the first sensed data FSD. For example, at least one of the physiological status and image is displayed in the frame FR and provided for the user 2 or the non-user to monitor the physiological status and/or image.

It is noteworthy that a unidirectional or bidirectional transmission of data may be conducted among the near-end portable monitoring module 12, the image sensing module 14 and the near-end information display module 16.

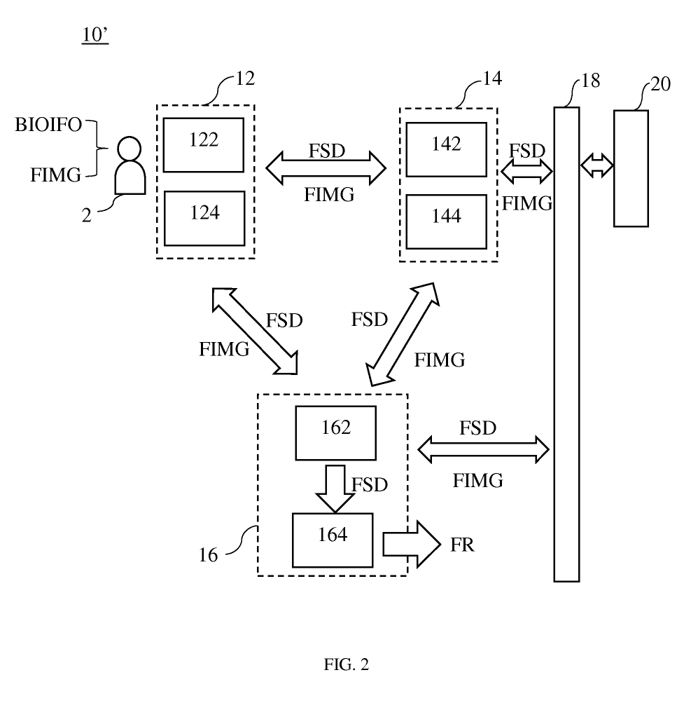

With reference to FIG. 2 for the schematic block diagram of a physiological monitoring system in accordance with the second preferred embodiment of the present invention, the physiological monitoring system 10′ further comprises a network communication unit 18 in addition to the near-end portable monitoring module 12, the image sensing module 14 and the near-end information display module 16 described in the first preferred embodiment.

The network communication unit 18 is connected to the Internet 20. The network communication unit 18 complies with a communication specification, and the communication specification also complies with at least one of the aforementioned wireless communication specification and cable communication specification. The network communication unit 18 may transmit the first sensed data FSD among the near-end portable monitoring module 12, the image sensing module 14 and the near-end information display module 16 via the Internet 20.

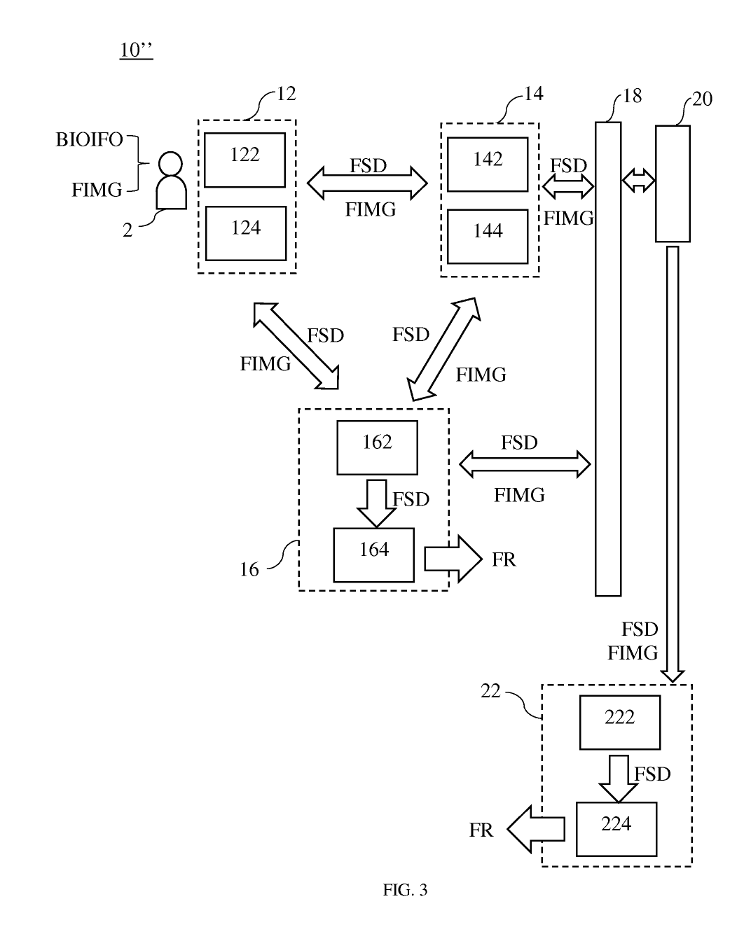

With reference to FIG. 3 for the schematic block diagram of a physiological monitoring system in accordance with the third preferred embodiment of the present invention, the physiological monitoring system 10″ further comprises a far-end information display module 22 in addition to the near-end portable monitoring module 12, the image sensing module 14, the near-end information display module 16, the network communication unit 18, and the Internet 20 as described in the second preferred embodiment.

The far-end information display module 22 comprises a fourth communication unit 222 and a second display unit 224. The fourth communication unit 222 receives the first sensed data FSD via the Internet 20 and displays the frame FR corresponsive to the first sensed data FSD on the second display unit 224. Wherein, the fourth communication unit 222 also complies with at least one of the aforementioned wireless communication specification and cable communication specification.

The far-end information display module 22 is connected to the Internet 20 directly or through the network communication unit 18 to receive the first sensed data FSD.

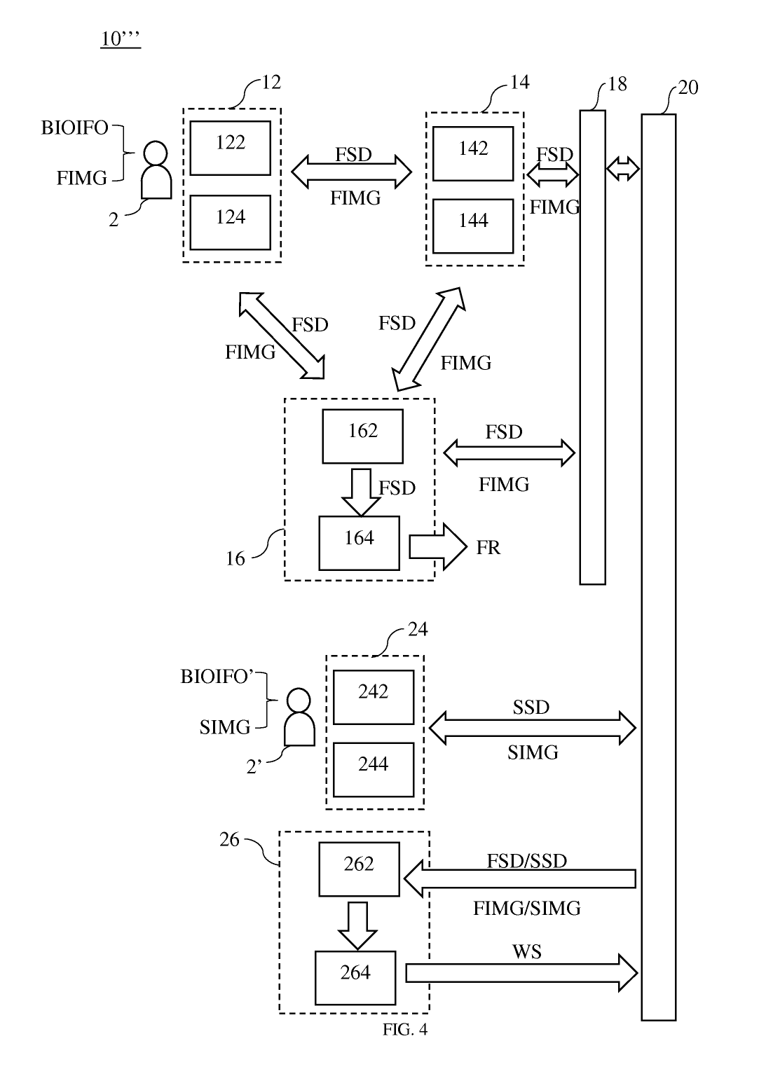

With reference to FIG. 4 for the schematic block diagram of a physiological monitoring system in accordance with the fourth preferred embodiment of the present invention, the physiological monitoring system 10″′ further comprises a far-end portable monitoring module 24 and an alarm unit 26 in addition to the near-end portable monitoring module 12, the image sensing module 14, the near-end information display module 16, the network communication unit 18, and the Internet 20 as described in the second preferred embodiment. The physiological monitoring system 10″′ is provided for monitoring at least one of the physiological status BIOIFO and the first image FIMG of a user 2 as well as monitoring at least one of the physiological status BIOIFO′ and the second image SIMG of another user 2′.

The far-end portable monitoring module 24 comprises a fifth communication unit 242 and a second sensing unit 244. The far-end portable monitoring module 24 is provided for the other user 2′ to wear. The second sensing unit 244 senses the physiological status BIOIFO′ and/or the second image SIMG of the other user 2′ to generate second sensed data SSD. The fifth communication unit 242 transmits at least one of the second sensed data SSD and the second image SIMG to the Internet 20. Wherein, the second sensing unit 244 is provided for sensing at least one selected from the group consisting of temperature, audio frequency, sound, humidity, brightness, movement and distance.

The alarm unit 26 comprises a sixth communication unit 262 and a first processing unit 264. The sixth communication unit 262 receives the second sensed data SSD, and the first processing unit 264 selectively generates a warning signal WS according to the second sensed data SSD. The warning signal WS is provided for warning an abnormal situation of the user 2 and the user 2′.

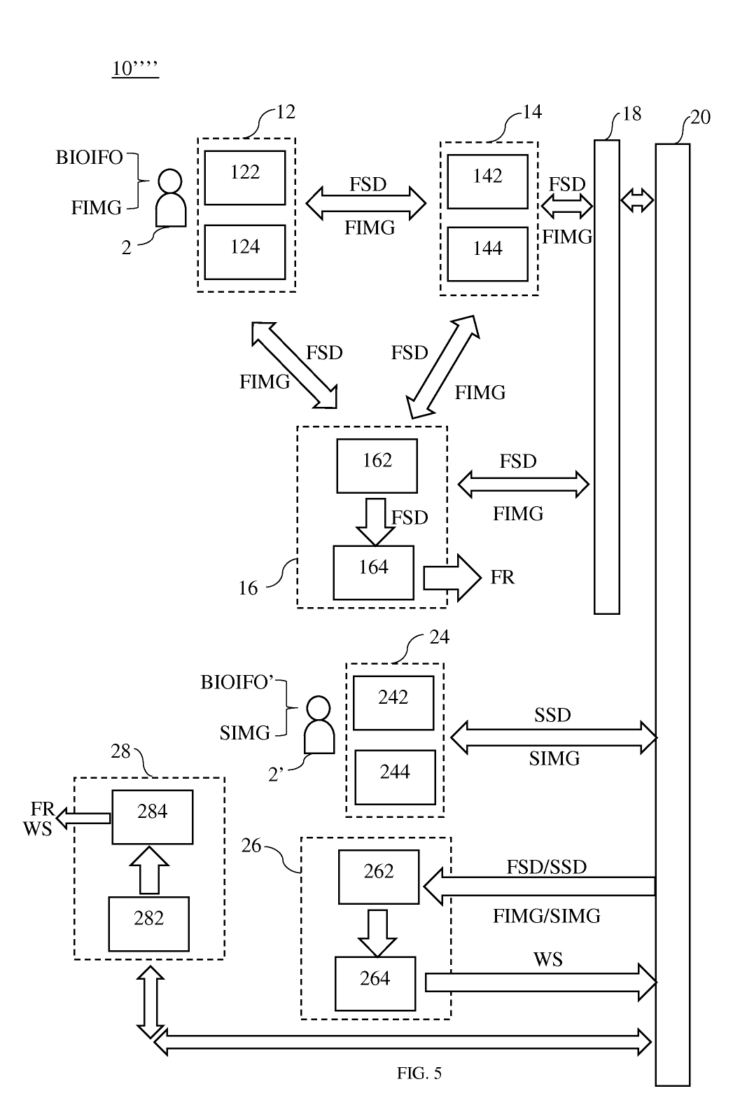

With reference to FIG. 5 for the schematic block diagram of a physiological monitoring system in accordance with the fifth preferred embodiment of the present invention, the physiological monitoring system 10″″ further comprises a servo host 28 in addition to the near-end portable monitoring module 12, the image sensing module 14, the near-end information display module 16, the network communication unit 18, the Internet 20, the far-end portable monitoring module 24 and the alarm unit 26 as described in the fourth preferred embodiment.

The servo host 28 comprises a seventh communication unit 282 and a second host unit 284. The seventh communication unit 282 receives at least one of the first sensed data FSD, the first image FIMG, the second sensed data SSD and the second image SIMG, and the second host unit 284 generates at least one frame FR according to the first image FIMG and the second image SIMG. In addition, the second host unit 284 generates a warning signal WS according to at least one of the first sensed data FSD and the second sensed data SSD.

While the invention has been described by means of specific embodiments, numerous modifications and variations could be made thereto by those skilled in the art without departing from the scope and spirit of the invention set forth in the claims.

What is claimed is:

1. A physiological monitoring system, for monitoring at least one of a user's physiological status and first image, comprising:
a near-end portable monitoring module, having a first communication unit and a first sensing unit, and provided for the user to wear, and the first sensing unit sensing the physiological status of the user to generate first sensed data, and the first communication unit transmitting the sensed data;
an image sensing module, having a second communication unit and an image capturing unit, and the second communication unit receiving the first sensed data, and the image capturing unit selectively capturing the first image of the user, wherein the image capturing unit tracks the user to obtain the first image by the near-end portable monitoring module; and
a near-end information display module, having a third communication unit and a first display unit, and the third communication unit receiving the first sensed data, and displaying a frame corresponsive to the first sensed data on the first display unit.

2. The physiological monitoring system as claimed in claim 1, wherein the first sensing unit is provided for sensing at least one selected from the group consisting of temperature, audio frequency, sound, humidity, brightness, movement and distance.

3. The physiological monitoring system as claimed in claim 1, further comprising a network communication unit for connecting the Internet, and the network communication unit complying with a communication specification, and at lease one of the first communication unit, the second communication unit and the third communication unit complying with the communication specification, so that the first sensed data are transmitted among the near-end portable monitoring module, the image sensing module and the near-end information display module via the Internet.

4. The physiological monitoring system as claimed in claim 3, further comprising a far-end information display module having a fourth communication unit and a second display unit, and the fourth communication unit receiving the first sensed data via the Internet and displaying the frame corresponsive to the first sensed data on the second display unit, wherein the fourth communication unit complies with the communication specification.

5. The physiological monitoring system as claimed in claim 3, further comprising a far-end portable monitoring module having a fifth communication unit and a second sensing unit, provided for the user to wear, and the second sensing unit sensing at least one of the physiological status and second image of another user to generate second sensed data, and the fifth communication unit transmitting the second sensed data to the Internet.

6. The physiological monitoring system as claimed in claim 5, wherein the second sensing unit is provided for sensing at least one selected from the group consisting of temperature, audio frequency, sound, humidity, brightness, movement and distance.

7. The physiological monitoring system as claimed in claim 5, further comprising an alarm unit having a sixth communication unit and a first processing unit, and the sixth communication unit receiving the second sensed data, and the first processing unit selectively generating a warning signal according to the second sensed data.

8. The physiological monitoring system as claimed in claim 5, further comprising a servo host having a seventh communication unit and a second host unit, and the seventh communication unit receiving at least one of the first sensed data, the first image, the second sensed data and the second image, and the second host unit generating at least one frame according to the first image and the second image, and the second host unit generating a warning signal according to at least one of the first sensed data and the second sensed data.

9. The physiological monitoring system as claimed in claim 1, further comprising an alarm unit having a sixth communication unit and a first processing unit, and the sixth communication unit receiving the first sensed data, and the processing unit selectively generating a warning signal according to the first sensed data.

10. The physiological monitoring system as claimed in claim 1, further comprising a servo host having a seventh communication unit and a second host unit, and the seventh communication unit receiving at least one of the first sensed data and the image, and the second host unit generating a frame according to the image, and the second host unit generating a warning signal according to at least one of the first sensed data and the image.
